(19) United States
(12) Patent Application Publication
(10) Pub. No.: US 2020/0113958 A1
(43) Pub. Date: Apr. 16, 2020

(54) Compositions and Methods For Balancing Body Acidity and Relieving Body Over Acidity Symptoms and Strengthening the Immune Response

(71) Applicant: Khaled Khalil, Shfaamr (IL)

(72) Inventor: Khaled Khalil, Shfaamr (IL)

(21) Appl. No.: 16/159,453

(22) Filed: Oct. 12, 2018

(51) Int. Cl. A61K 36/28; A61K 47/36; A61K 9/00; A61K 36/31; A61P 39/00; A61P 31/16

(52) U.S. Cl. CPC A61K 36/28; A61K 47/36; A61P 31/16; A61K 36/31; A61P 39/00; A61K 9/0053

(57) ABSTRACT

The present invention relates to methods and compositions effective in balancing over acidity levels or treating, preventing, or relieving the symptoms of, body over acidity or a condition associated with influenza. The compositions comprise an extract from the plant genera Taraxacum as an active ingredient, preferably from the roots of the species Taraxacum officinale. The composition can also be administered in combination with a traditional anti-over acidity medicine, either over-the-counter or prescription medicines in treating over acidity, or in combination with an extract of Sinapis arvensis seeds in treating influenza. The compositions are particularly effective in treating and preventing over acidity, the symptoms or conditions associated with over acidity.

### FIELD OF THE INVENTION

The present invention relates to a pharmaceutical composition comprising an extract from the plant Dandelion and its use in balancing body over acidity, and reducing the severity of, or relieving the symptoms of immune system deficiency that related with body over acidity. The plant roots are the preferred source of the extracts. The present invention also relates to the pharmaceutical composition that is administered in combination with a traditional anti-over acidity medicine, either over-the-counter or prescription medicines in treating over acidity, or to the pharmaceutical composition in combination with an extract of Sinapis arvensis in treating influenza.

### BACKGROUND OF THE INVENTION

### Background of Deficiency

Acidosis means that kidneys and lungs can't keep body's pH in balance. Problems with these organs can lead to excess acid accumulating in our body. According to the American Association for Clinical Chemistry (AACC), The normal pH of blood should be around 7.4. Acidosis is characterized by a pH of 7.35 or lower. Alkalosis is characterized by a pH level of 7.45 or higher. Acidic body is very common today, especially in people suffer from different diseases.

Over acidity in the body can cause problems such as cardiovascular damage, including the constriction of blood vessels and the reduction of oxygen; weight gain, obesity and diabetes; bladder and kidney conditions, including kidney stones; immune deficiency; acceleration of free radical damage, possibly contributing to cancerous mutations; premature aging; osteoporosis, weak, brittle bones, hip fractures and bone spurs; joint pain, aching muscles and lactic acid buildup; and low energy and chronic fatigue.

### Over Acidity and Immune Response

By having a bodily pH outside of the optimal range, we begin to see a reduction in the molecular movements of white blood cells toward the invading pathogens. Therefore, the overall immune response is weakened when an excessive extracellular pH imbalance is present. Over acidity condition forces the body to borrow minerals—including calcium, sodium, potassium and magnesium—from organs and bones to neutralize the acid and remove it from the body.

Buffer systems

The three main buffer systems in our body are: protein buffer system, phosphate buffer system and bicarbonate buffer system. Each of these buffers has different mechanism to neutralize the pH level.

(I) Protein Buffer System Mechanism

The protein hemoglobin can bind to small amounts of acid in the blood, helping to remove that acid before it changes the blood's pH. Many other proteins act as buffers as well. Proteins containing the amino acid histidine are particularly adept at buffering.

(II) Phosphate Buffer System Mechanism

The phosphate buffer system is comprised of two ions: hydrogen phosphate ions and dihydrogen phosphate ions. The pH level of the blood drops below 7.4 when the H+ ions in the bloodstream increase. Hydrogen phosphate ions accept all additional H+ ions to re-establish the equilibrium between the hydroxide and hydrogen ions in the blood. When the pH level of the blood increases above 7.4, the dihydrogen phosphate ions release additional hydrogen ions to reinstate the pH level of the blood to its optimal 7.4.

(III) Bicarbonate Buffer System Mechanism

The bicarbonate buffer system functions to maintain the pH level in the blood of mammals. It also plays a major role in the formation of acid in the stomach, and to neutralize the pH of chyme that enters the small intestine from the stomach. The bicarbonate buffer system manages acid/base imbalances and effectively manages the release of excess carbon dioxide as a bi-product of cellular respiration. It is conceptual that minerals and different substances in the body assist the buffer systems maintain pH level.

### Treatment for Acidosis

Usually treatments with sodium bicarbonate can be used for any type of acidosis. This can be done either by mouth or in an intravenous (IV) drip to raise the pH of the blood. The treatment for other types of acidosis can involve treating their cause. Treatments for Respiratory acidosis are usually designed to help lungs functioning. Like giving drugs to dilate airway, or giving oxygen or a continuous positive airway pressure (CPAP) device. The CPAP device can help in breathing if patient has an obstructed airway or muscle weakness. Patients with hyperchloremic acidosis may be given oral sodium bicarbonate. Acidosis from kidney failure may be treated with sodium citrate. Diabetics with ketoacidosis receive IV fluids and insulin to balance out their pH. Lactic acidosis treatment might include bicarbonate supplements, IV fluids, oxygen, or antibiotics, depending on the cause.

### Background of Influenza

According to the World Health Organization (WHO) “Influenza is a viral infection that affects mainly the nose, throat, bronchi and, occasionally, lungs. Infection usually lasts for about a week, and is characterized by sudden onset of high fever, aching muscles, headache and severe malaise, non-productive cough, sore throat and rhinitis.”

The virus is transmitted easily from person to person via droplets and small particles produced when infected people cough or sneeze. Influenza tends to spread rapidly in seasonal epidemics. Most infected people recover within one to two weeks without requiring medical treatment. However, in the very young, the elderly, and those with other serious medical conditions, infection can lead to severe complications of the underlying condition, including pneumonia and death. Influenza (also called flu) is a contagious respiratory illness caused by influenza viruses. It can cause mild to severe illness. The familiar way to prevent the flu is by getting a flu vaccination each year. According to the data of Centers for Disease Control and Prevention (CDC), every year in the USA, on average 5% to 20% of the population gets the flu; more than 200,000 people are hospitalized from flu complications, and about 36,000 people die from flu.

### Background of the Plants

This invention deals with compositions comprising extracts from plant belonging to the genera Taraxacum or specifically to Taraxacum officinale. The preferred species commonly known as dandelions,which is native to Eurasia and North America, but the two commonplace species worldwide, T. officinale and T. erythrospermum, were introduced from Europe and now propagate as wildflowers.

Dandelion plant is familiar to therapeutic use, it has been used for a long time for its properties and support for liver complaints (Schütz et al., 2006; Domitrovié et al., 2010). Dandelion has been used to improve energy levels and health (Lee et al., 2012). Research has shown that compounds in plants have pharmacokinetic effects (Rodriguez-Fragoso et al., 2008). Dandelion contains antioxidant and redness-resistant compounds (González-Castejón et al., 2012). Research showed that dandelion positively affected antioxidant enzyme activities and lipid profiles (Jeon et al., 2008). Researchers determine that dandelion could protect against oxidative stress related to certain circulatory disorders (Choi et al., 2010).

Because dandelion helps to stimulate bile production and bile transport toxins out of the body, dandelion root may support your body's natural immune response. A study devised to compare the activity of artichoke, dandelion, turmeric and rosemary extracts found their antioxidant activity to support the rationale the traditional inclusion of these ingredients in gallbladder applications (Menghini et al., 2010). A study found that dandelion extract demonstrated antioxidant activity against free radicals to counteract acetaminophen liver toxicity (Colle et al., 2012). Animal studies have been successful in finding beneficial links between certain liver problems and dandelion (You et al., 2010).

The present invention also relates to compositions comprising extracts from plants belonging to the genera Sinapis and Brassica, such as mustards. The preferred species is Sinapis arvensis, but any species from the above genera is suitable. The extracts are preferably obtained from the plant's seeds. Sinapis arvensis is an annual or overwintering herb with a stale and unpleasant odor. It has a slender tap root and a usually erect stiff, little branched hairy stem, 10-60 cm in height. Lower leaves are stalked, upper leaves are more or less sessile and all are pinnatisect or rarely pinnate, with a long oblong or obovate terminal lobe which is coarsely toothed or lobed, rarely entire. Leaves appear very similar in appearance to dandelion. Flowers are relatively large, few and in small terminal racemes. Petals are 12-20 mm, pale yellow or whitish with deep violet veins, twice as long as the sepals. Fruits are a cylindrical siliqua about three times as broad as long [(12-25)×(3-5) mm], erect and more or less parallel to the stem on short erect stalks and with a flattened beak. Seeds are 1.5-2 mm, yellow-brown or reddish, spherical or ovoid in 2 rows in each cell. Sinapis arvensis is a native of southern Europe and central Asia where it has been cultivated since historical times. The plants are naturalized in waste places, road shoulders and fallow fields in northern and western Europe, well beyond their original habitat. The seed extract of Sinapis arvensis contains inter alia linoleic acid, oleic acid and erucic acid.

### Dandelion Active Ingredients

Bitter glycosides (taraxacin), tannins, triterpenes (including taraxol and taraxsterol), phytosterols, volatile oil, choline, asparagine, carbohydrates (including inulin, up to 40% in autumn, 2% in spring; sugars), pectin, phenolic acids, vitamins, and potassium are the active ingredients in the dandelion.

In every 100 gm of fresh dandelion there are plenty of nutrients and minerals (Chhabra, accessed in 2018), which are as follows:
vitamins (mg/100g of fresh leaves): vitamin A 14,000, thiamine 0.19, riboflavin 0.26, niacin 0.0, and vitamin C 35;
minerals (mg/100g of fresh leaves): calcium 187, Phosphorus 66, iron 3.1, sodium 76, and potassium 397; and
other nutrients (mg/100g of fresh leaves): calories 45, protein 2.7, fat 0.7, and carbohydrates 9.2.

### Dandelion Proposed Mechanism of Action

Dandelion extract contains minerals (mg/100g of fresh leaves) such as calcium 187, phosphorus 66, iron 3.1, sodium 76 and potassium 397. All of these minerals are used by the buffering system to increase pH level in the case of acidic pH. The buffer system may use other substances that the body produces to balance pH level.

Iron has important role to oxygenate our body and helps to make functional hemoglobin, protein that is found in red blood cells. In our lungs, hemoglobin binds to oxygen molecules from the air we breathe, and releases that oxygen into our tissues. Proper oxygen transport in our body partially relies on calcium. Calcium has an important role in oxygenation by controlling blood vessel dilation—it can dilate blood vessels around tissues that need more oxygen at any given time to provide more oxygen-rich blood flow, or constrict blood vessels and reduce blood flow to tissues that need less oxygen. Getting enough calcium might also help keep the circulatory system healthy as you age. It is known that calcium reduces the risk of high blood pressure. Copper also helps tissues get a sufficient supply of oxygen. And also helps the body incorporate iron into hemoglobin. In this state of functionality, hemoglobin can bind to small amounts of acid in the blood, helping to remove that acid before it changes the blood's pH. Potassium level that is too high causes hyperkalemia and if too low causes hypokalemia, both are serious. Thus, potassium in case of acidic body assists the buffers to increase pH level.

We assume that Dandelion extract helps the buffering system to function well when the body acidity is high. In other words, Dandelion extract improves functionality of immune response, as buffer systems are part of immune system. We conclude that Dandelion extract could be used as treatment of body acidity due to its active ingredients which include minerals needed for increasing pH level. It eases the functionality of the buffer systems and support them to use more efficiently to provide these ingredients as a result of taking the extract, instead of providing the needed components from the body systems only.

### SUMMARY OF THE INVENTION

The present invention relates to methods and compositions effective in balancing body over acidity, and reducing the severity of, or relieving the symptoms of immune system deficiency that related with body over acidity, as an active ingredient, an extract from the plant genus Taraxacum, preferably from roots of the plant species Taraxacum officinale such as Dandelion. The composition comprises only natural ingredients and has been demonstrated to be safe for use, while still providing a beneficial therapeutic effect. The composition can also be administered in combination with traditional medicines, either over-the counter or prescription medicines. The compositions are particularly effective in treating and preventing immune response related to acidosis, the symptoms or and conditions associated with this deficiency.

The present invention also relates to pharmaceutical compositions comprising an extract from the plant genera Sinapis and Brassica, preferably from the species Sinapis arvensis, and methods of treating, preventing, reducing the severity of, or relieving the symptoms of influenza and associated conditions by administering the composition comprising the extract from the plant of Sinapis arvensis, or the extract from the plant of Sinapis arvensis and the extract from the Dandelion. The plant seeds are the preferred source of the extracts.

### BRIEF DESCRIPTION OF THE DRAWINGS

FIGS. 1A and 1B describe the production process of Dandelion extracts.

### DETAILED DESCRIPTION OF THE INVENTION

The anti over acidity compositions of this invention comprise an extract of a plant from the genera Taraxacum, with preference Taraxacum officinale such as Dandelion. The roots of the plant are the preferred source of the extracts. The plant has traditionally been used in the treatment of several diseases and infections, but was never used or tested against the body over acidity, which is caused by various conditions. Our trials on patients confirm the effectiveness of compositions containing Dandelion extract in treating the over acidity within a comparatively short span of time in comparison to chemical drugs, without any significant side effects.

The present invention relates to methods and compositions effective in treating, preventing, reducing the severity of, or relieving the symptoms of over acidity. The compositions comprise, as an active ingredient, an extract from the roots of the plant genus Taraxacum, preferably from the species Taraxacum officinale. The composition comprises only natural ingredients and has been demonstrated to be safe for use, while still providing a beneficial therapeutic effect. The compositions are particularly effective in treating and preventing over acidity, the symptoms or in over acidity and conditions associated with over acidity.

The compositions of the present invention offer significant advantages over current state-of-the-art body over acidity treatment options, as they contain only natural ingredients and are thus safe for use. Therefore, use of the compositions of the invention will reduce or completely eliminate the unpleasant side effects typically associated with traditional or conventional anti-over acidity medicines as further discussed below, while still achieving a beneficial therapeutic effect. Therefore, patient compliance may be substantially improved. Accordingly, the present invention provides a significant improvement over the current-state-of-the-art pertaining to over acidity treatment. The present invention thus provides, in one embodiment, a method of treating, preventing, reducing the severity of, or relieving the symptoms of over acidity and conditions associated with over acidity, comprising administering to a subject in need thereof a composition comprising, an extract from Dandelion roots, in an amount effective to treat, prevent, reduce the severity of, or relieve the symptoms of, over acidity or conditions associated with over acidity.

In another aspect, the present invention provides pharmaceutical compositions useful for treating, preventing, reducing the severity of, or relieving the symptoms of over acidity and conditions associated with over acidity, the compositions comprising an extract from Dandelion roots, in an amount effective to treat, prevent, reduce the severity of, or relieve the symptoms of, over acidity or conditions associated with over acidity; and a pharmaceutically acceptable carrier or excipient.

In another aspect, the present invention relates to a method of manufacturing a medicament from an extract of a Dandelion for treating, preventing, reducing the severity of, or relieving the symptoms of over acidity and conditions associated with over acidity.

Any plant of the Taraxacum genera can be used in the present invention. The preferred plant species is Taraxacum officinale and the part of the plant preferred as source for the extracts and compositions of this invention is its roots.

In a currently preferred embodiment, an extract from a Dandelion species is used. In accordance with this embodiment, the present invention provides a method of treating, preventing, reducing the severity of, or relieving the symptoms of body over acidity and conditions associated with over acidity, comprising administering to a subject in need thereof a composition comprising an extract from Dandelion, in an amount effective to treat, prevent, reduce the severity of, or relieve the symptoms of, over acidity or conditions associated with over acidity.

Though not wishing to be bound by any particular mechanism or theory, it is contemplated that the plant extract of the present invention is effective at treating the symptoms of over acidity, by balancing pH levels and affecting the function of immune system.

In one embodiment, the Dandelion extract is a solid, dried extract. In another embodiment, the extract is an aqueous extract. In another embodiment, the extract is an alcoholic extract. The extract is provided in an amount effective to treat, prevent, reduce the severity of, or relieve the symptoms of, over acidity or conditions associated with over acidity. The recommended daily dose is 1800 mg of dried extract, recommended for three months at least.

The composition typically comprises from about 10% to about 90% of the plant extract, for example from about 20% to about 80% plant extract, from about 30% to about 70% plant extract. Currently preferred concentration of plant extract is about 40% or about 50%. All percentages are expressed herein as weight percentages based on the total weight of the composition.

In some embodiments, the Dandelion extract of the present invention can be taken alone, for example when the symptoms of over acidity or associated conditions are noticed, or on a regular regimen as a natural supplement. In other embodiments, however, the extract can be taken together with the one or more additional active ingredients which are traditional anti-over acidity medicines, either over-the-counter or prescription medicines.

The Dandelion extract and the at least one other agent can be formulated in the same composition, or they can be in different compositions. If administered in separate compositions, the Dandelion extract and the at least one other agent can be administered sequentially or concurrently, in any order. It is contemplated that the concurrent use of the plant extract of the present invention together with the traditional anti-over acidity medicines, will lower the dose of the traditional medicines that is required in order to reach a therapeutic effect, resulting in decreased side effects and higher patient compliance.

The present invention relates, in one embodiment, to pharmaceutical compositions comprising a Dandelion extract, and at least one pharmaceutically acceptable carrier or excipient, for example fillers, diluents, binders, disintegrating agents, surfactants, lubricants, solvents, stabilizers, solubilizers, tonicity enhancing agents, buffers, preservatives, thickeners, complexing agents, emulsifiers, antioxidants, flavor-improving agents, gellants, glidants, sweeteners, colorants, viscosity increasing agents, and any combinations thereof.

A currently preferred excipient is a maltodextrin. In a specific embodiment, the composition comprises a Dandelion extract and maltodextrin.

The compositions of the invention can be administered via oral, topical, sublingual, buccal, parenteral, paracentral, intravenous, transdermal, inhalation, intranasal, vaginal, intramuscular, transmucosal, transdermal, rectal, intradermal, subcutaneous, intraperitoneal, intraventricular, intracranial, rectal, vaginal, or intratumoral administration.

Suitable oral administration forms include, but are not limited to, a tablet, caplet, capsule (e.g., soft or hard gelatin capsule), microcapsule, pellet, pill, powder, syrup, gel, slurry, granule, suspension, dispersion, emulsion, liquid, solution, dragée, bead and beadlet.

The compositions of the present invention are natural compositions that are highly effective in treating, balancing, reducing the severity of body over acidity, and strenthening the immune resposse and relieving the symptoms of over acidity and associated conditions. The compositions are safe to use and are particularly advantageous due to the reduction or elimination of side effect that are associated with conventional anti-acidity drugs currently on the market.

A fixed concentrated dry extract of Dandelion was assessed efficacy in reducing body over acidity levels. The extract of the plant dry powder was encapsulated with supplements of maltodextrin and so called Acidlevel capsules of 450 mg each. A clinical study was carried out by general physicians in Israel.

### Material and Methods

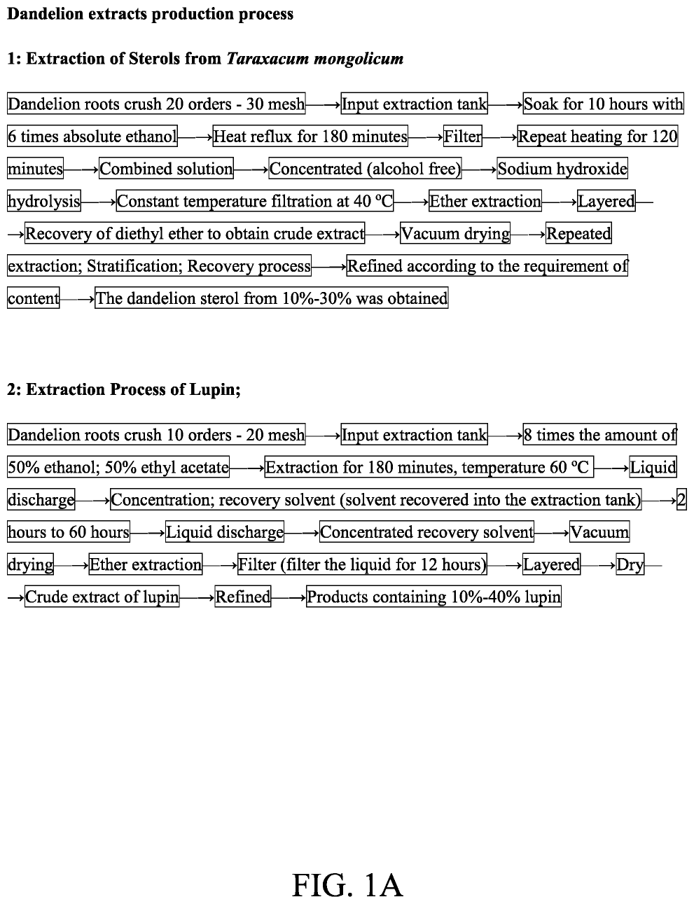

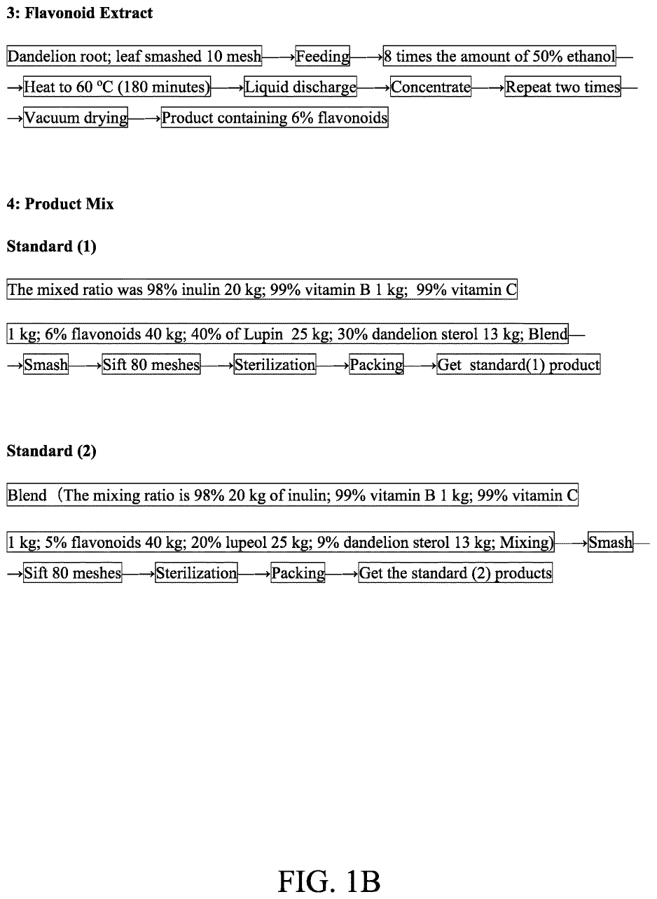

The roots of dandelion were extracted by a professional extraction company according to methodology of double standardization as it shown in FIGS. 1A and 1B.

### Clinical Investigation for Treating Over Acidity (Salivary Test)

### Patient Selection and Clinical Protocol:

Patients were selected on the basis of routine visits to Biomed KK clinic in Shefa amr, north of Israel, they were motivated to take herbal therapy to maintain a healthy acids level in the body. After a thorough review of Acidlevel-components, they were asked to continue their daily activities and food habits unchanged and to take 2 capsules of Acidlevel 2 times daily before meal at morning and after meal at evening. All other usual medications were kept unchanged during the study period of 3 months. Each patient had to give an oral consent and was given a free-of-charge box containing 360 capsules of Acidlevel.

We have studied 20 individuals with different disorders and diseases: 5 with cancer, 7 with diabetes type 2, and 8 with hypertension. The 20 participants in the study had two total spontaneous saliva samples collected in the morning after a 12-hour fasting period, one in the pre-treatment phase and another in post treatment. The patients were instructed not to use toothpaste on the harvesting day.

Salivary pH was digitally measured through a digital pH sensor. We initially calibrated the device using buffered solutions with pH 4.0 and pH 7.0. After that the sensor probe was dipped in saliva filled Falcon tube, where it remained for 30 seconds, thus yielding automatic pH reading.

### Dandelion Capsules Treatment

Treatment was initially clinical and standardized for all patients, being: Dandelion 900 mg per before breakfast and 900 mg after dinner for 12 (twelve) weeks. After this time, the patients' saliva were harvested 2 days after medication interruption. We compared both the saliva before and after treatment. Results were put in a table shown below (Table I) and statistically analyzed.

### Inclusion Criteria

We only included in the study those patients that had diagnosis of cancer (abbreviated as CA), diabetes type 2 (DT2), and hypertension (HT).

### Exclusion Criteria

Exclusion factors were smoking and alcohol drinking.

### Results

A total of 20 patients were eligible to participate in the study, 12 women and 8 men, ranging from 19 to 56 years. The average pre-treatment salivary pH of the group was 6.25 and post-treatment was 7.155, statistically significant difference (p<0.05).

TABLE I
Salivary pH data from the participants in the study.

| Patient # | Gender | Age | disease | PH Pre | PH post |
|---|---|---|---|---|---|
| 1 | M | 56 | DT2 | 6.4 | 7.3 |
| 2 | F | 60 | HT | 6.1 | 7.2 |
| 3 | M | 46 | DT2 | 6.3 | 6.9 |
| 4 | F | 53 | CA | 6 | 6.9 |
| 5 | M | 24 | CA | 6.1 | 7.4 |
| 6 | F | 19 | CA | 6.1 | 7.1 |
| 7 | F | 41 | HT | 6.5 | 7.2 |
| 8 | M | 43 | DT2 | 6.4 | 7.1 |
| 9 | F | 36 | CA | 5.8 | 7.4 |
| 10 | F | 50 | HT | 6.2 | 7.4 |
| 11 | F | 21 | CA | 6 | 7.0 |
| 12 | F | 44 | DT2 | 5.9 | 6.7 |
| 13 | F | 56 | DT2 | 6.3 | 7.4 |
| 14 | F | 47 | HT | 5.9 | 7.4 |
| 15 | M | 50 | HT | 6.9 | 7.3 |
| 16 | M | 48 | DT2 | 6.7 | 7.1 |
| 17 | F | 48 | HT | 6.5 | 7.4 |
| 18 | M | 46 | HT | 6.8 | 7.2 |
| 19 | M | 48 | DT2 | 6.1 | 6.8 |
| 20 | F | 47 | HT | 6.0 | 6.9 |

### Discussion

The results disclose that Acidlevel is therapeutically efficient as substantial and incremental reductions of over acidity levels were observed before and after 3 months. At 3 months, baseline acid levels in these patients were significantly changed and became normal. We propose that the Dandelion component of Acidlevel is in accord with recommendations in traditional herbalism and has primarily effect that supports the liver and immune system functioning.

### Over Acidity Treatment

The Dandelion roots extract can be provided in a solid, dried extract form or in a liquid form. The extract can be provided alone, or mixed with one or more inert excipients. In one embodiment, the extract is provided as a solid (dried) extract. In another embodiment, the extract provided in a liquid form. In another embodiment, the extract is provided as an aqueous extract. In another embodiment, the extract is provided an alcoholic extract. The procedure is exemplified in the Examples and FIGS. 1A and 1B.

In a preferred embodiment, the extract is obtained by extraction of the Dandelion roots with a mixture of 50% ethanol and 50% water. This extract may be optionally dried.

The final product, whether solid or liquid, can comprise from about 1% to about 100% extract, the balance being made up of inert excipients. For example, the product can comprise from about 10% to about 90% extract, from about 20% to about 80% extract, from about 30% to about 70% extract, from about 40% to about 60% extract, and the like. In one currently preferred embodiment, the product comprises about 40% extract and about 60% inert excipient(s). In another currently preferred embodiment, the product comprises about 50% extract and about 50% inert excipient(s). All percentages recited herein refer to weight/weight (wt/wt) percentages, based on the total weight of the composition.

The extract is provided in an amount effective to treat, prevent, reduce the severity of, or relieve the symptoms of, over acidity or conditions associated with over acidity.

### Clinical Investigations for Treating Influenza

### The Selection of Volunteers and Clinical Protocol

Fifty eight of human volunteers (aged 18-65) were selected on the basis that they were suffering from either the common cold or influenza. The volunteers were not taking any pharmacological agents. They were recruited from two general physician clinics in the Galilee in the north of Israel and were willing to take a herbal therapy. After a thorough review of the components of the compositions of this invention they were asked to continue their normal daily activities and habits and to take one capsule containing extracts of Sinapis arvensis seeds three times per day. They were also asked to refrain from using any other medications during the one-week study period. The subjects gave their informed consent and each was given a box containing 21(450 mg each) capsules of composition free of charge.

### Results

Fifty four of the patients experienced reduced fever from the third day of using the capsules and started to feel well and revitalized on the fourth day, with suffering from mild pain and influenza symptoms. All patients took the capsules for three more days as a preventative measure. Four patients did not react.

### Pharmaceutical Compositions

Although the active extracts of the invention can be administered alone, it is contemplated that the extract will be administered in a pharmaceutical composition containing the active ingredient together with a pharmaceutically acceptable carrier or excipient.

The Dandelion extracts used in the Examples were prepared by extraction of Dandelion roots with a mixture of 50% ethanol and 50% water as detailed in Example 4 part B. Alternatively, extracts from other plants of the Taraxacum genus may be used. Thus, in one embodiment, the present invention provides pharmaceutical compositions useful for treating, preventing, reducing the severity of, or relieving the symptoms of over acidity and conditions associated with over acidity, the compositions comprising a dandelion or Taraxacum roots extract, in an amount effective to treat, prevent, reduce the severity of, or relieve the symptoms of, over acidity or conditions associated with over acidity; and a pharmaceutically acceptable carrier or excipient.

Pharmaceutical compositions for use in accordance with the present invention can be formulated in conventional manner using one or more physiologically acceptable carriers comprising excipients and auxiliaries, which facilitate processing of the active compounds into preparations which can be used pharmaceutically. The active agents are formulated as pharmaceutical compositions and administered to a mammalian subject, such as a human patient in a variety of forms such as liquid, solid, and semisolid. The pharmaceutical compositions can be administered to a subject by any method known to a person skilled in the art, such as oral, topical, sublingual, buccal, parenteral, intravenous, transdermal, inhalation, intranasal, vaginal, intramuscular, transmucosal, transdermal, rectal, intradermal, subcutaneous, rectal, or vaginal administration.

For oral administration, the compounds can be formulated by combining the Dandelion or Taraxacum extract with pharmaceutically acceptable carriers known in the art. The compositions can be formulated in any solid or liquid dosage form known in the art, including but not limited to, tablet, caplet, capsule (e.g., a soft or hard gelatin capsule), microcapsule, pellet, pill, powder, syrup, gel, slurry, granule, suspension, dispersion, emulsion, liquid, solution, dragée, bead and beadlet. The oral compositions can be formulated as immediate release formulations, or as controlled or sustained release formulations allowing for extended release of the active ingredient(s) over a predetermined time period.

Suitable excipients for solid formulations include but are not limited to fillers (diluents) such as sugars, including lactose, sucrose, mannitol, or sorbitol; starch based excipients such as maize starch, wheat starch, rice starch, potato starch and the like; gelatin, gum tragacanth; phosphates such as calcium phosphate (e.g. dicalcium phosphate, tricalcium phosphate); cellulose based excipients as microcrystalline cellulose, carboxymethylcellulose, hydroxymethylcellulose, hydroxyethylcellulose, methylhydroxypropylcellulose, hydroxypropylcellulose and the like. Polymers such as polyvinylpyrrolidone (PVP) and cross-lined PVP can also be used. In addition, the compositions may further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. corn starch, potato starch, alginic acid, silicon dioxide, croscarmellose sodium, crospovidone, guar gum, sodium starch glycolate), surfactants (e.g. sodium lauryl sulfate), and lubricants (e.g. stearic acid, magnesium stearate, polyethylene glycol, sodium lauryl sulfate).

In another embodiment, the composition further comprises at least one vitamin. Examples of suitable vitamins include, but are not limited to, vitamin A, vitamin B, vitamin C, vitamin D, vitamin E, and mixtures thereof.

For liquid formulations, pharmaceutically acceptable carriers may be aqueous or non-aqueous solutions, suspensions, emulsions or oils. Examples of nonaqueous solvents are propylene glycol, polyethylene glycol, and injectable organic esters. Aqueous carriers include water, alcoholic/aqueous solutions, emulsions or suspensions, including saline and buffered media. Examples of oils include but are not limited to petroleum, animal, vegetable, or synthetic origin, for example, peanut oil, soybean oil, mineral oil, olive oil, sunflower oil, and fish-liver oil.

Preferred oral pharmaceutical compositions include capsules made of gelatin as well as soft, sealed capsules made of gelatin and a plasticizer, such as glycerol or sorbitol. In soft capsules, the active compounds may be dissolved or suspended in suitable liquids, such as fatty oils, liquid paraffin, or liquid polyethylene glycols. In addition, stabilizers may be added. In certain preferred embodiments the capsules exclude components of animal origin and are acceptable for vegetarians and vegans.

Soft gelatin capsules and methods of preparing them are known in the art. Non-limiting examples can be found in U.S. Pat. No. 6,217,902 (Tanner et al., 2001); U.S. Pat. No. 6,258,380 (Overholt 2001); U.S. Pat. No. 5,916,591 (Bierdel-Willkommen et al., 1999), and U.S. Pat. No. 4,891,229 (Brox et al., 1990), all of which are incorporated herein by reference.

Other acceptable excipients and additives known to the person skilled in the art may be included in the compositions of the present invention, for example, stabilizers, solubilizers, tonicity enhancing agents, buffer substances, preservatives, thickeners, anti-oxidants, complexing agents, flavor-improving agents such as sweeteners, e.g., aspartame, gellants, glidants, colorants and viscosity-increasing agents. A solubilizer can be for example, tyloxapol, fatty acid glycerol polyethylene glycol esters, fatty acid polyethylene glycol esters, polyethylene glycols, glycerol ethers or mixtures of those compounds. A specific example of a solubilizer is a polyoxyethylated castor oil for example, the commercial products Cremophor® or Cremophor® RH40. Another example of a solubilizer is tyloxapol. The concentration used depends especially on the concentration of the active ingredient. The amount added is typically sufficient to solubilize the active ingredient. For example, the concentration of the solubilizer is from 0.1 to 5000 times the concentration of the active ingredient.

Examples of buffer substances are acetate, ascorbate, borate, hydrogen carbonate/carbonate, citrate, gluconate, lactate, phosphate, propionate and TRIS (tromethamine) buffers. The amount of buffer substance added is, for example, that necessary to ensure and maintain a physiologically tolerable pH range. The pH range is typically in the range of from 5 to 9, preferably from 5.2 to 8.5.

Tonicity enhancing agents are selected from ionic and non-ionic agents. For example, ionic compounds include alkali metal or alkaline earth metal halides, such as, for example, CaCl₂, KBr, KCl , LiCl , Nal, NaBr or NaCl, or boric acid. Non-ionic tonicity enhancing agents are, for example, urea, glycerol, sorbitol, mannitol, propylene glycol, or dextrose.

Examples of preservatives are quaternary ammonium salts such as benzalkonium chloride, benzoxonium chloride or polymeric quaternary ammonium salts, alkyl-mercury salts of thiosalicylic acid, such as thiomersal, phenylmercuric nitrate, phenylmercuric acetate or phenylmercuric borate; parabens such as methylparaben or propylparaben; alcohols such as chlorobutanol, benzyl alcohol or phenyl ethanol; guanidine derivatives such as chlorohexidine or polyhexamethylene biguanide, or sorbic acid. Where appropriate, a sufficient amount of preservative is added to the composition to ensure protection against secondary contaminations during use caused by microbes. Where appropriate, anti-oxidants are also added.

The compositions of the present invention may further comprise non-toxic excipients, such as emulsifiers, wetting agents (surfactants) or fillers, for example, the polyethylene glycols (PEG200, 300, 400 and 600) or Carbowax® (Carbowax1000, 1500, 4000, 6000 and 10000). Other excipients that may be used if desired are listed below but they are not intended to limit in any way the scope of the possible excipients. They can be complexing agents, such as disodium-EDTA or EDTA; antioxidants, such as ascorbic acid, acetylcysteine, cysteine, sodium hydrogen sulfite, butyl-hydroxyanisole, butyl-hydroxytoluene; stabilizers, such as thiourea, thiosorbitol, sodium dioctyl sulfosuccinate or monothioglycerol; or other excipients, such as, for example, lauric acid sorbitol ester, triethanol amine oleate or palmitic acid ester.

The amount and type of excipient added is in accordance with the particular requirements and is generally in the range of from approximately 1% to approximately 99% by weight.

The amount of a composition to be administered will, of course, depend on many factors including the subject being treated, the severity of the affliction, the manner of administration, and the judgment of the prescribing physician. However, the dose employed will generally depend on a number of factors, including the age and sex of the patient, and the severity of the disease being treated.

Preferably, the preparations are in unit dosage form, intended for oral administration. In such form, the preparation is subdivided into unit doses containing appropriate quantities of the active components. The unit dosage form can be a packaged preparation, the package containing discrete quantities of preparation, for example, tablets, capsules, and powders in vials or ampoules. The unit dosage form can also be a capsule, cachet, or tablet itself or it can be the appropriate number of any of these in packaged form.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

The Dandelion extracts used in the examples below were prepared by extraction of

Dandelion roots with a mixture of 50% ethanol and 50% water as detailed in Example 4, part B

### Example 1

### Oral Compositions

A 450 mg capsule comprising a Dandelion roots extract was prepared. The capsule comprises 230 mg concentrated Dandelion extract, 220 mg maltodextrin. The maltodextrin was added as part of the drying process during extraction. This method of drying may be replaced by other methods such as spray drying.

### Example 2

### Toxicity of Dandelion Extract

The material safety data sheet (MSDS) of extract of Dandelion roots have shown high level of safety. For example, the extract contains ≤10 ppm of total heavy metals, ≤0.5 ppm of arsenic, ≤2 ppm of lead, ≤1 ppm of cadmium, and ≤0.5 ppm of mercury; zero amount of sulfate, organic phosphate, organic residues or pesticide residues; no E. coli, Salmonella and Staphylococcus.

### Example 3

### Clinical Investigations for Treating Over Acidity

Patients were selected on the basis of routine visits to Biomed KK clinic in Shefa amr, north of Israel, they were motivated to take herbal therapy to maintain a healthy acids level in the body. After a thorough review of Acidlevel-components, they were asked to continue their daily activities and food habits unchanged and to take 2 capsules of Acidlevel 2 times daily before meal at morning and after meal at evening. All other usual medications were kept unchanged during the study period of 3 months. Each patient had to give an oral consent and was given a free-of-charge box containing 360 capsules of Acidlevel. The results were in Table I shown above.

A total of 20 patients were eligible to participate in the study, 12 women and 8 men, ranging from 19 to 56 years. The average pre-treatment salivary pH of the group was 6.25 and post-treatment was 7.155, statistically significant difference (p<0.05).

### Example 4

### Extract Preparation for Dandelion

Analytical Procedures: The plant extracts were analyzed by HPLC and UV & VIS spectroscopy.

Reagents: Acetonitrile: Water 70:30. All reagents used are HPLC grade.

Sample Preparation:

A. Extraction with 99% Ethanol

Fifty kg of milled plant was extracted with 130 kg of 99% ethyl alcohol for 2.5 hours at a temperature of 70° C., then filtered through a fine filter. The residue was again extracted using another 130 kg of 99% ethyl alcohol and filtered. The filtrates from first and second extraction were mixed and evaporated. 240 kg of the filtrate at a starting total dissolved solids (TDS) percentage of 0.81% was evaporated up to 30% TDS at a temperature less than 60° C. with a final weight of 8 Kg. The product can be supplied in a liquid form or dried with a carrier (e.g., maltodextrin). In this case the final product consists of 50% active material and 50% inert carrier.

B. Extraction with a Mixture of 50% Ethanol and 50% Water

Fifty kg of milled plant was extracted with 233 kg mixture of 50% ethanol and 50% water for 2.5 hours at a temperature of 70° C., then filtered through a fine filter. The residue was again extracted using another 50 kg mixture of 50% ethanol and 50% water and filtered. The filtrates from the first and second extraction were mixed and evaporated, providing a dried extract, which was used for the preparation of the compositions of this invention. 350 kg of the filtrate with a starting TDS of 1.7% was evaporated up to 30% TDS at a temperature less than 60° C. with a final weight of 5.95 kg. The product can be supplied in a liquid form or dried with a carrier (e.g., maltodextrin). In this case the final product consists of 50% active material and 50% inert carrier.

C. Extraction with Water

Fifteen kg of milled plant was extracted with 70 kg water for 2.5 hours at a temperature of 70° C., then filtered through a fine filter. The residue was again extracted using another 50 kg water and filtered. The filtrates from extractions 1 and 2 were mixed and evaporated. 100 kg of the filtrate at a starting TDS of 2.1% was evaporated up to 30% TDS at a temperature less than 60° C. with a final weight of 7 kg. The product can be supplied in a liquid form or dried with a carrier (e.g., maltodextrin). In this case the final product consists of 50% active material and 50% inert carrier.

### Example 5

### Extract Preparation for Sinapis arvensis

Analytical Procedures: The plant extracts were analyzed by HPLC and UV & VIS spectroscopy.

Reagents: Acetonitrile: Water 70:30. All reagents used are HPLC grade.

Sample Preparation:

A. Extraction with 99% Ethanol

Fifteen kg of milled plant was extracted with 40 kg of 99% ethyl alcohol for 2.5 hours at a temperature of 70° C., then filtered through a fine filter. The residue was again extracted using another 40 kg of ethyl alcohol 99% and filtered. The filtrates from first and second extraction were mixed and evaporated. 69 kg of the filtrate at a starting total dissolved solids (TDS) percentage of 0.81% was evaporated up to 30% TDS at a temperature less than 60° C. with a final weight of 2.3 Kg. The product can be supplied in a liquid form or dried with a carrier (e.g., TCP). In this case the final product consists of 50% active material and 50% inert carrier.

B. Extraction with a Mixture of 50% Ethanol and 50% Water

Fifteen kg of milled plant was extracted with 70 kg mixture of 50% ethanol and 50% water for 2.5 hours at a temperature of 70° C., then filtered through a fine filter. The residue was again extracted using another 50 kg mixture of 50% ethanol and 50% water and filtered. The filtrates from the first and second extraction were mixed and evaporated, providing a dried extract, which was used for the preparation of the compositions of this invention. 105 kg of the filtrate with a starting TDS of 1.7% was evaporated up to 30% TDS at a temperature less than 60 “C with a final weight of 5.95 kg. The product can be supplied in a liquid form or dried with a carrier (e.g., TCP). In this case the final product consists of 50% active material and 50% inert carrier.

C. Extraction with Water

Fifteen kg of milled plant was extracted with 70 kg water for 2.5 hours at a temperature of 70° C., then filtered through a fine filter. The residue was again extracted using another 50 kg water and filtered. The filtrates from extractions 1 and 2 were mixed and evaporated. 100 kg of the filtrate at a starting TDS of 2.1% was evaporated up to 30% TDS at a temperature less than 60° C. with a final weight of 7 kg. The product can be supplied in a liquid form or dried with a carrier (e.g., TCP). In this case the final product consists of 50% active material and 50% inert carrier.

### Example 6

### Clinical Investigation for Treating Influenza Using Extracts of Mustard Seeds and Dandelion Roots.

Twenty two human volunteers (aged 18-65) were selected on the basis that they were suffering from either the common cold or influenza. The volunteers were not taking any pharmacological agents. They were recruited from two general physician clinics in the Galilee in the north of Israel and were willing to take a herbal therapy. After a thorough review of the components of the compositions of this invention they were asked to continue their normal daily activities and habits and to take 2 Dandelion capsules twice per day and 2 Mustard capsules twice per day. They were also asked to refrain from using any other medications during the one-week study period. The subjects gave their informed consent and each was given a box containing 14 capsules of Dandelion (450 mg each) and a box of 14 capsules of Mustard (450 mg each) free of charge. It has been found that 19 of the patients experienced reduced fever from the first and the second day of using the capsules and started to feel well and revitalized on the third day, without suffering from pain or any other influenza symptoms. All patients took the capsules for three more days as a preventative measure. 3 patients did not react.

### Conclusion

We conclude that the compositions of the two plants may have synergistic effect in treating influenza. The results show significant effect from the first and second day, while the effect in the mustard investigation started from the third day.

As used herein, “a” or “an” means one or more (or at least one).

As used herein, “about” means in the range of ±10%.

Although the invention has been explained in relation to its preferred embodiment, it is to be understood that many other possible modifications and variations can be made without departing from the spirit and scope of the invention.

What is claimed is:

1. A method of treating, preventing, reducing the severity of, or relieving the symptoms of, over acidity or a condition associated with influenza, comprising administering to a subject in need thereof a composition comprising an extract from a plant of Taraxacum genus or Dandelion, in an amount effective to treat, prevent, reduce the severity of, or relieve the symptoms of, over acidity or a condition associated with influenza.

2. The method according to claim 1, wherein the extract is from a Taraxacum officinale species.

3. The method according to claim 1, wherein the extract is from the roots of a Taraxacum officinale plant.

4. The method of claim 1, wherein at least one of the symptoms of over acidity is immune response deficiency.

5. The method of claim 1, wherein the composition comprises the extract in solid form or in liquid form.

6. The method of claim 1, wherein the composition comprises a water or an alcohol extract of Taraxacum officinale.

7. The method of claim 1, wherein the extract is obtained from the roots of the Taraxacum officinale plant by extraction with a mixture of 50% ethanol and 50% water and is subsequently dried.

8. The method of claim 1, wherein the composition comprises from about 10% to about 90% by weight of the Taraxacum officinale extract.

9. The method of claim 1, wherein the composition comprises from about 30% to about 70% by weight of the Taraxacum officinale extract.

10. The method of claim 1, wherein the composition comprises about 40% or about 50% by weight of the Taraxacum officinale extract.

11. The method of claim 1, wherein the extract possesses anti-oxidant activity.

12. The method of claim 1, wherein the composition further comprises at least one pharmaceutically acceptable carrier or excipient.

13. The method of claim 12, wherein the at least one pharmaceutically acceptable carrier or excipient is selected from the group consisting of fillers, diluents, binders, disintegrating agents, surfactants, lubricants, solvents, stabilizers, solubilizers, tonicity enhancing agents, buffers, preservatives, thickeners, complexing agents, emulsifiers, antioxidants, flavor-improving agents, gellants, glidants, sweeteners, colorants, viscosity-increasing agents, and any combinations thereof.

14. The method of claim 12, wherein the excipient is maltodextrin.

15. The method of claim 14, wherein the composition comprises about 40% by weight of the Taraxacum officinale extract, and about 60% by weight of maltodextrin.

16. The method of claim 14, wherein the composition comprises about 50% by weight of the Taraxacum officinale extract, and about 50% by weight of maltodextrin.

17. The method of claim 1, wherein the composition is administered via oral, topical, sublingual, buccal, parenteral, intravenous, transdermal, inhalation, intranasal, vaginal, intramuscular, transmucosal, transdermal, intradermal, subcutaneous, or rectal administration.

18. The method according to claim 1, wherein the composition is administered orally.

19. The method according to claim 1, wherein the composition is in a form selected from the group consisting of a tablet, caplet, capsule, microcapsule, pellet, pill, powder, syrup, gel, slurry, granule, suspension, dispersion, emulsion, liquid, solution, dragée, bead and beadlet.

20. The method of claim 19, wherein the capsule is selected from a soft gelatin capsule and a hard gelatin capsule.

21. The method of claim 1, wherein the composition is administered once daily, twice-daily, thrice daily, once weekly or once monthly.

22. The method of claim 1, wherein the composition is administered as a needed regimen to balance pH levels and relieve the symptoms of over acidity or a condition associated with over acidity.

23. The method of claim 1, wherein the composition is administered in combination with at least one other agent.

24. The method of claim 23, where the at least one other agent is a traditional anti-over acidity medicine.

25. The method of claim 23, where the at least one other agent is an extract from Sinapis arvensis to treat a condition associated with influenza.

26. A process for manufacturing an extract of Taraxacum officinale, comprising extracting the Taraxacum officinale roots with a solvent comprising alcohol, water or mixtures thereof.
